(19) United States
(12) Patent Application Publication
(10) Pub. No.: US 2022/0117799 A1
(43) Pub. Date: Apr. 21, 2022

(54) SYSTEM AND METHOD FOR A NURSING PAD

(71) Applicant: KEWE LLC d/b/a Modern Mom Intimates, Denver, CO (US)

(72) Inventor: Kelly Elizabeth Westergren, Denver, CO (US)

(73) Assignee: KEWE LLC d/b/a Modern Mom Intimates, Denver, CO (US)

(21) Appl. No.: 17/505,508

(22) Filed: Oct. 19, 2021

Related U.S. Application Data
(60) Provisional application No. 63/093,708, filed on Oct. 19, 2020.

(51) Int. Cl. A61F 13/14

(52) U.S. Cl. CPC A61F 13/141; A61F 2013/15016

(57) ABSTRACT

A nursing pad includes an absorbent pad and an adhesive. The absorbent pad defines a first side and a second side and has a contoured shaped configured to correspond to a shape of a user's breast. The adhesive is attached to the second side of the absorbent pad and is configured to adhere the absorbent pad to the user's breast.

### TECHNICAL FIELD

The present disclosure relates generally to absorbent articles, and more particularly relates to nursing pads, and related methods for using nursing pads.

### BACKGROUND

Nursing pads are typically worn by nursing mothers to absorb breast milk that has leaked from the mother's breast. The pads are typically applied to the mother's breast Nursing pads are typically worn by nursing mothers to absorb breast milk that has leaked from the mother's breast. The pads are typically applied to the mother's breast and maintained in position by a garment or article of clothing such as a bra. At least some known nursing pads include an adhesive on a surface of the nursing pad that faces away from the mother's breast when applied to the mother's breast. The adhesive adheres to the bra in order to maintain the nursing pad in position during use. However, the nursing pad may still move relative to the mother's breast because the adhesive that adheres the nursing pad to the bra may be inadequate and may lead to the nursing pad bunching up within the bra, causing discomfort and leakage. Additionally, at least some known nursing pads are made with undesirable materials, such as non-organic materials, that may provoke allergic reactions, not allow breathability, may not be absorbent, and may not be biodegradable or compostable. Moreover, at least some known nursing pads are flat and not contoured to the shape of the user's breast. The flat shape of the nursing pad does not allow the nursing pad to conform to the user's breast when the nursing pad is positioned against the breast. Further, the pads do not have a means of securing to the breast or being fixed in place. As a result, there is slippage and leakage.

### SUMMARY

One aspect of the present disclosure relates to a stick on, peel off disposable nursing pad including an absorbent pad and an adhesive. The absorbent pad defines a first side and a second side and has a contoured shaped configured to correspond to a shape of a user's breast. The adhesive is attached to the second side of the absorbent pad and is configured to adhere the absorbent pad directly to the user's breast.

Another aspect of the present disclosure relates to a nursing pad including an absorbent pad and an adhesive. The absorbent pad defining a first side and a second side and is formed of a plurality of layers of organic cotton. The adhesive may be attached to the second side of the absorbent pad and is configured to adhere the absorbent pad to the user's breast.

Another aspect of the present disclosure relates to a method of using a nursing pad. The method includes providing the nursing pad including an absorbent pad and an adhesive. The adhesive includes an adhesive substance and a backing. The method also includes removing the backing to expose the adhesive substance. The method further includes applying the nursing pad to a user's breast by adhering the adhesive substance to the user's breast.

Yet another aspect of the present disclosure relates to a method of making a nursing pad. The method includes applying an adhesive to a first quilted layer of an absorbent pad. The method also includes applying a backing onto the adhesive to protect the area of the adhesive before use. The method also includes layering at least one additional layer of an absorbent pad on the first quilted layer of an absorbent pad. The method also includes applying a barrier layer on the at least one additional layer of the absorbent pad. The method also includes compressing the first quilted layer of an absorbent pad, the at least one additional layer of an absorbent pad, and the barrier layer together.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### DRAWINGS

Implementations are disclosed in association with the accompanying drawings in which:

FIG. 1 illustrates a perspective view of an absorbent article in accordance with aspects in the present disclosure.

FIG. 2 illustrates a front view of the absorbent article shown in FIG. 1 in accordance with aspects in the present disclosure.

FIG. 3 illustrates a front view of the absorbent article shown in FIG. 1 with a backing removed from the absorbent article in accordance with aspects in the present disclosure.

FIG. 4 illustrates a back view of the absorbent article shown in FIG. 1 in accordance with aspects in the present disclosure.

FIG. 5 illustrates a schematic side view of the layers of the absorbent article shown in FIG. 1 in accordance with aspects in the present disclosure.

FIG. 6 illustrates a schematic front view of the absorbent article shown in FIG. 1 in accordance with aspects in the present disclosure.

FIG. 7 is a flow diagram of a method of using the absorbent article shown in FIG. 1 in accordance with aspects in the present disclosure.

### DETAILED DESCRIPTION

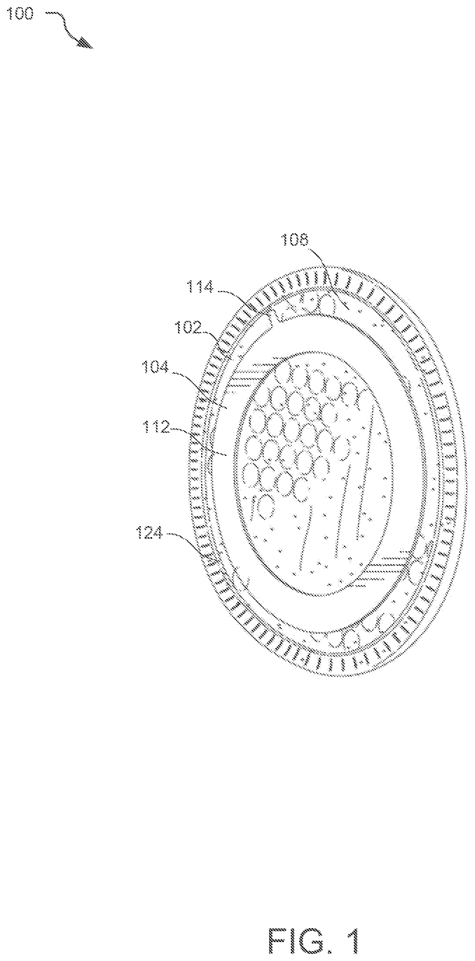

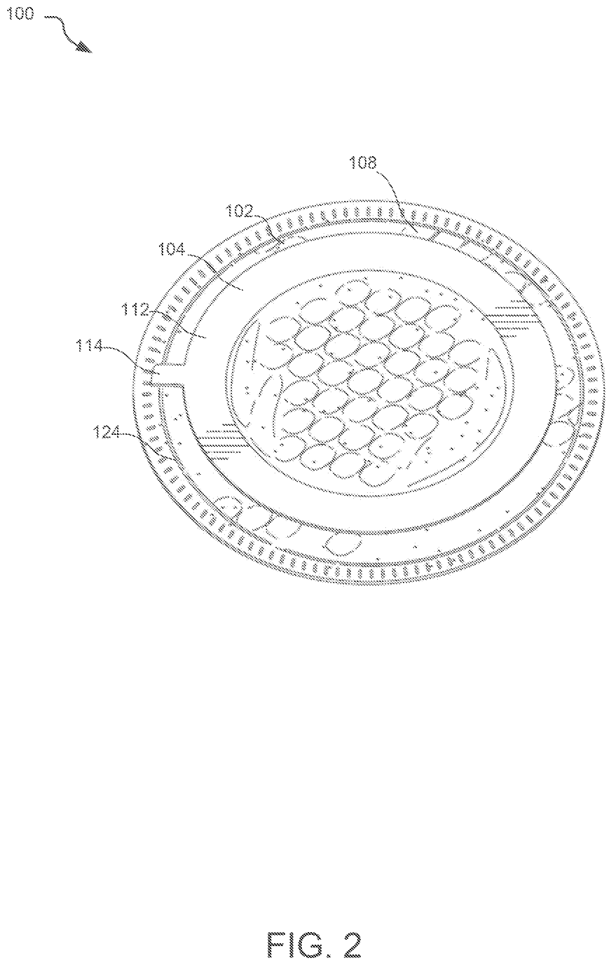

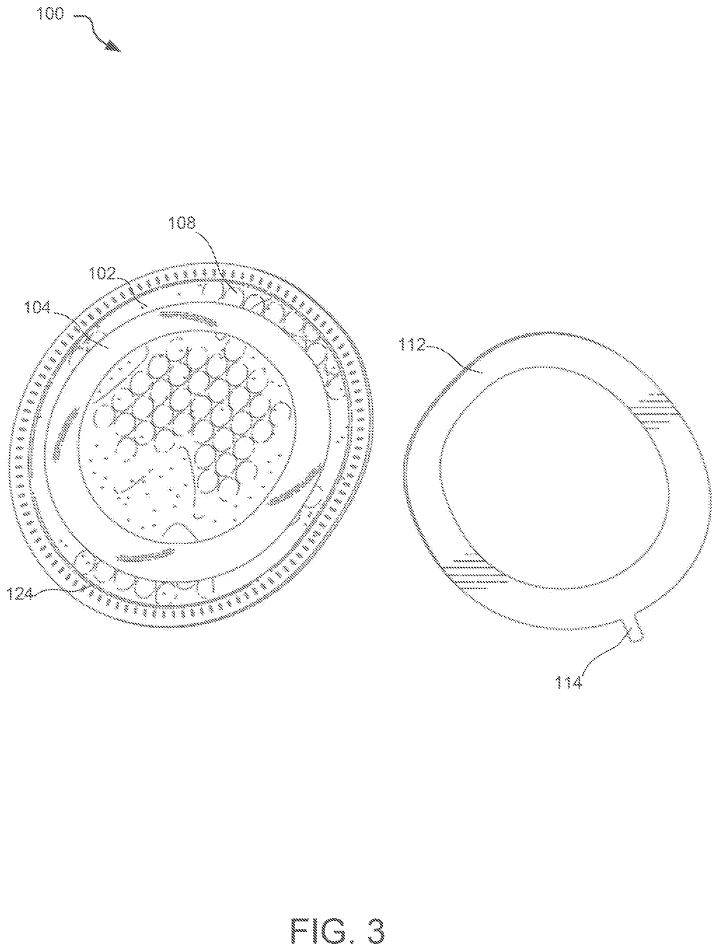

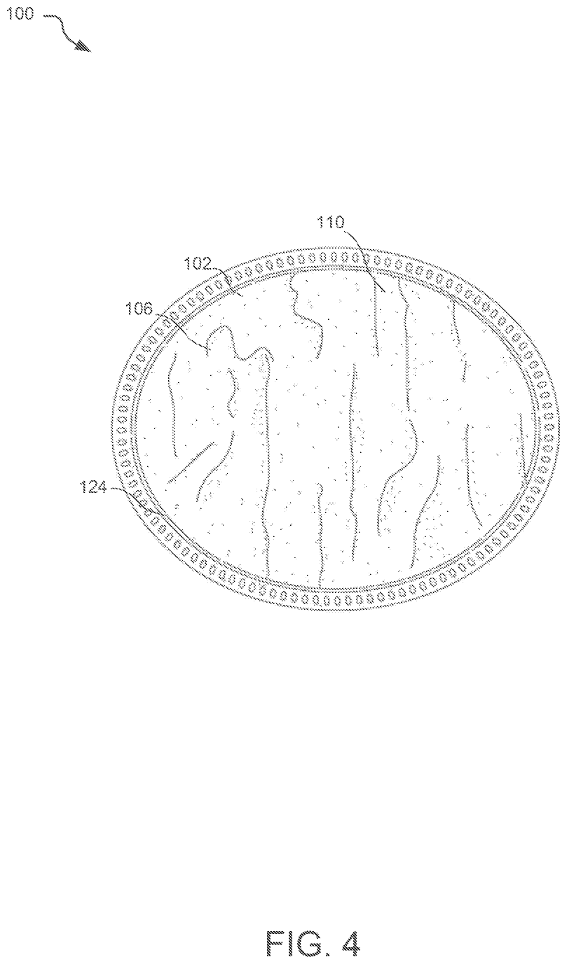

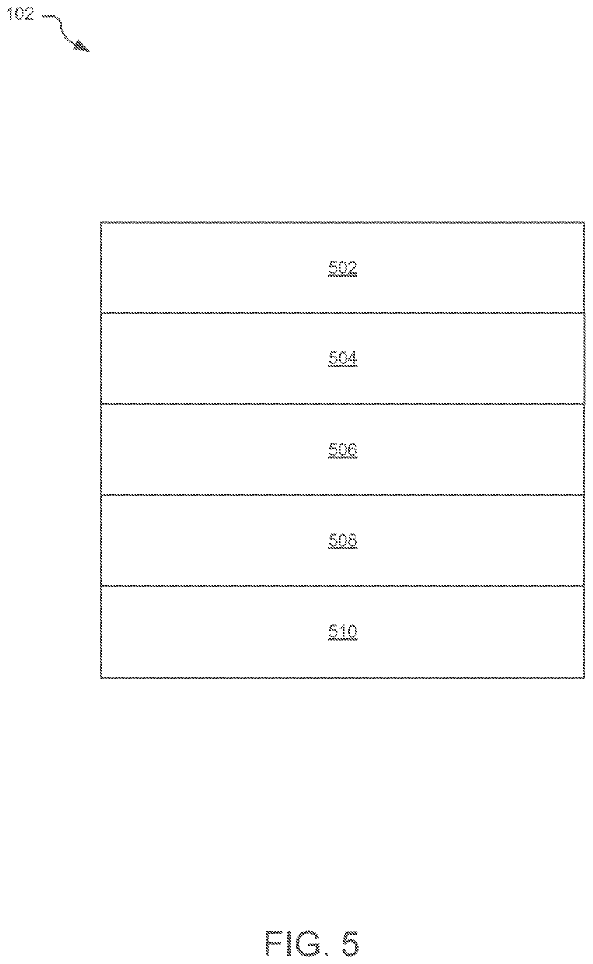

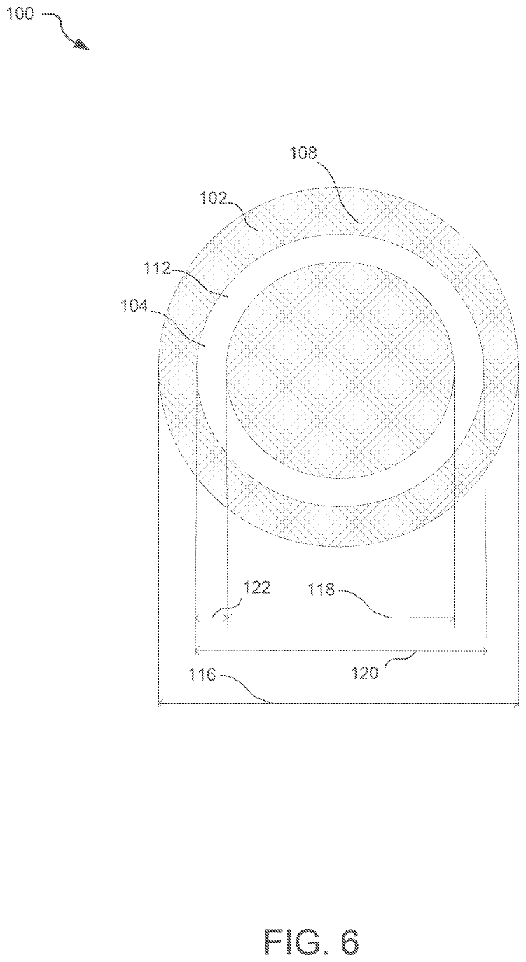

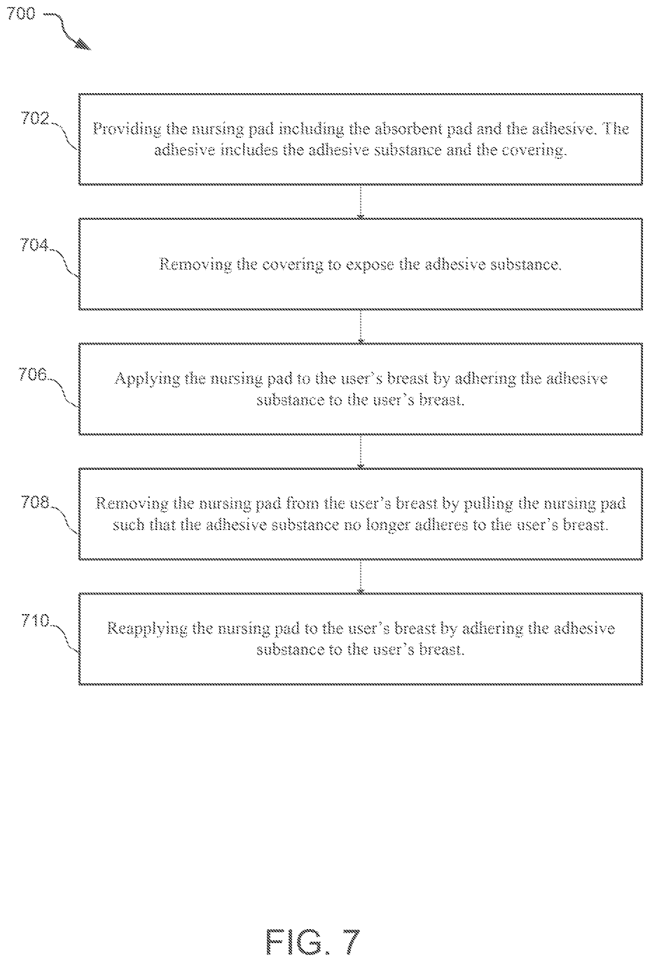

FIG. 1 illustrates a perspective view of an absorbent article in accordance with aspects in the present disclosure. FIG. 2 illustrates a front view of the absorbent article shown in FIG. 1 in accordance with aspects in the present disclosure. FIG. 3 illustrates a front view of the absorbent article shown in FIG. 1 with a backing removed from the absorbent article in accordance with aspects in the present disclosure. FIG. 4 illustrates a back view of the absorbent article shown in FIG. 1 in accordance with aspects in the present disclosure. FIG. 5 illustrates a schematic side view of the layers of the absorbent article shown in FIG. 1 in accordance with aspects in the present disclosure. FIG. 6 illustrates a schematic front view of the absorbent article shown in FIG. 1 in accordance with aspects in the present disclosure. FIG. 7 is a flow diagram of a method of using the absorbent article shown in FIG. 1 in accordance with aspects in the present disclosure.

In the illustrated embodiments, the absorbent article 100 includes a nursing pad 100. In alternative embodiments, the absorbent article 100 may be any type of absorbent article including, without limitation, bandages, feminine hygiene products, diapers, sanitary articles, and/or any other article configured to absorb liquid.

In the illustrated embodiment, the nursing pad 100 includes an absorbent pad 102 and an adhesive 104 attached to the absorbent pad 102. The absorbent pad 102 is configured to be attached to a user's breast by the adhesive 104 and to absorb liquid (breast milk) produced or leaked from the user's breast. Specifically, the absorbent pad 102 includes a first side 108 and a second side 106. The adhesive 104 is attached to the first side 108. The user exposes the adhesive 104 and applies the first side 108 of the absorbent pad 102 to the user's breast such that the adhesive 104 maintains the absorbent pad 102 in place on the user's breast. The user can remove the absorbent pad 102 from the user's breast by peeling the absorbent pad 102 away from the user's breast such that the adhesive 104 no long adheres the absorbent pad 102 to the user's breast. Accordingly, the nursing pads 100 described herein enable the absorbent pad 102 to be adhered to a user's breast without additional support from clothing (e.g., bras or other supportive articles of clothing) and improves the absorbency of the absorbent pad 102 by adhering the absorbent pad 102 directly to the user's breast.

The absorbent pad 102 is formed from a plurality of layers (shown and described in FIG. 5) of an absorbent material. In the illustrated embodiment, the absorbent material includes cotton. Specifically, in the illustrated embodiment, the absorbent material includes organic cotton. More specifically, in the illustrated embodiment, the absorbent material includes certified organic cotton that is hypoallergenic and biodegradable. In one implementations, the organic cotton used to manufacture the absorbent pad 102 has been certified as organic by the Istituto per la Certificazione Etica ed Ambientale (ICEA) and typically has been awarded the ICEA NR Certificate GOTS 2010-009. The organic cotton used to manufacture the absorbent pad 102 has been without the use of pesticides, herbicides, and/or chemical fertilizers. Additionally, no perfumes, chlorine, dioxin, and/or plastics were used in the cotton purifying process such that the absorbent pad 102 is hypoallergenic and at least 74% compostable and biodegradable. In some embodiments, the absorbent pad 102 may be 100% compostable and biodegradable.

In the illustrated embodiment, the layers of absorbent material each include a layer of absorbent material formed in a quilted pattern on the first side 108 of the absorbent pad 102 to provide breathability and to pull or wick moisture away from the user's breast and maintain the integrity of the adhesive 104. In some embodiments, the layers of absorbent material are formed into a waffle pattern to support the adhesive 104 on the first side 108 of the absorbent pad 102. In some embodiments, the quilted or waffle pattern provides additional support for the adhesive 104 such that the adhesive substance remains on the absorbent pad 102 when the nursing pad 100 is removed from and reapplied to the user's breast. Additionally, in order to reduce leaks, the absorbent pad 102 may include a barrier layer 110 positioned on the second side 106 of the absorbent pad 102. The barrier layer 110 may be formed of various materials configured to act as a vapor barrier, a liquid barrier, and/or a liquid and vapor barrier.

The absorbent pad 102 is formed by compressing the layers of absorbent material and the barrier layer 110 (if included) until the layers from a cohesive absorbent pad 102. Specifically, in the illustrated embodiment, the layers of absorbent material are stacked on the barrier layer 110 (if included) until the predetermined thickness of the absorbent pad 102 is achieved. The layers are then compressed by a compression machine or manually such that the layers from the cohesive absorbent pad 102. In some implementations, the compression machine compresses the layers such that the outer diameter of the absorbent pad 102 has a seal 124 that prevents liquid from escaping the absorbent pad 102. Additionally, the compression machine may cut the layers into the shape of the absorbent pad 102. In alternative embodiments, the layers may be stitched together to form the seal 124 rather than compressed to form the seal 124.

The nursing pad 100 includes the barrier layer 110, a plurality of intermediate layers of absorbent material, and an inner layer to be in contact with the skin of the user. The layers of the nursing pad 100 may include a variety of materials. For example, the barrier layer may be formed of biodegradable vapor barriers. In some implementations, the intermediate layers of absorbent material may include a hydrophilic cotton core formed by layers of the certified organic cotton, or other material, in order to improve the distribution or dispersion of the liquids absorbed by the absorbent pad 102, optimizing the absorption capacity of the nursing pad 100. In some implementations, the inner layer is formed of the quilted certified organic cotton. The hydrophilic cotton core wicks moisture away, reducing clogs that may develop because of stagnant liquid near the user's breast.

As shown in FIG. 1, the absorbent pad 102 has an ergonomic, anatomical shape that conforms to the shape of the user's breast. Specifically, the compression machine or person making the absorbent pad 102 forms the absorbent pad 102 into the ergonomic, anatomical shape during the compression process such that the absorbent pad 102 has a curved, contoured shape that corresponds to the shape of the user's breast. The ergonomic, anatomical shape enables the adhesive 104 to properly adhere the absorbent pad 102 and reduce leakage of liquids. More specifically, in the illustrated embodiment, the absorbent pad 102 has a circular, concave shape that forms a cup configured to receive the user's breast. In alternative embodiments, the absorbent pad 102 may have any shape that enables the nursing pad 100 to operate as described herein.

The adhesive 104 includes an adhesive substance (not shown) attached to the first side 108 of the absorbent pad 102 and a backing 112 selectively attached to the adhesive substance. In the illustrated embodiment, the adhesive substance includes a medical grade adhesive. In alternative embodiments, the adhesive substance may be any type of adhesive that enables the nursing pad 100 to operate as described herein. In another alternative embodiment, the nursing pad 100 may not include the adhesive 104. Rather, the absorbent pad 102 may be maintained on the user's breast with an article of clothing (e.g., a bra).

The backing 112 covers and protects the adhesive substance prior to use to maintain the adhesive substances ability to bond the absorbent pad 102 to the user's breast when not in use. The backing 112 has a shape that corresponds to the shape of the adhesive substance on the first side 108 of the absorbent pad 102. In the illustrated embodiment, the adhesive substance is formed in an adhesive ring and the backing 112 has a corresponding ring shape. In alternative embodiments, the adhesive substance and the backing 112 may have any shape that enables the nursing pad 100 to operate as described herein. In the illustrated embodiment, the backing 112 includes a wax paper covering including a pull tab 114 for removal of the backing 112. In alternative embodiments, the backing 112 may be any type of covering that enables the nursing pad 100 to operate as described herein.

The compressed organic cotton absorbent pad 102 maintains the adhesive substance on the absorbent pad 102 during use. In some implementations, the compressed organic cotton absorbent pad 102 provides a robust substrate that retains the adhesive substance on the absorbent pad 102 so that the absorbent pad 102 may be repeatedly applied to and removed from the user's breast, for example, if a user needs to remove the absorbent pad 102 to nurse. Further, the compressed organic cotton absorbent pad 102 enables the nursing pad 100 to be applied and removed to and from the user's breast multiple times throughout the use of the nursing pad 100 without the adhesive substance adhering to the user's breast.

As shown in FIG. 5, the absorbent pad 102 includes a plurality of layers 502-510 including a first layer 502, a second layer 504, a third layer 506, a fourth layer 508, and a fifth layer 510. In the illustrated embodiment, the first layer 502 includes the quilted inner layer configured to be in contact with the skin of the user and the adhesive 104 attached to the quilted inner layer. The second layer 504, third layer 506, and the fourth layer 508 include the intermediate layers that may or may not be quilted. The fifth layer 510 includes the barrier layer 110.

As shown in FIG. 6, the measurements of the absorbent pad 102 can vary. In some implementations, the absorbent pad 102 has an absorbent pad diameter 116 and the adhesive 104 has an inner diameter 118, an outer diameter 120, and a width 122. In the illustrated embodiment, the absorbent pad diameter 116 is about 7.62 centimeters (cm) to about 12.7 cm, about 8.89 cm to about 11.43 cm, or about 10.16 cm. The outer diameter 120 is about 7 cm to about 9 cm, about 7.5 cm to about 8.5 cm, or about 8 cm. The inner diameter 118 is about 6 cm to about 8 cm, about 6.5 cm to about 7.5 cm, or about 7 cm. The width 122 is about 0.5 cm to about 2 cm, about 0.75 cm to about 1.5 cm, or about 1 cm.

In some implementations, a plurality of nursing pads 100 may be stored and transported in a container. In some examples, the container includes a bag. In alternative embodiments, the container may include any other container including, without limitation, a box or a tube. Additionally, the container may be stored or transported in another container. Moreover, the nursing pads 100 may be individually wrapped in the container. In some embodiments, the container may be configured to store and transport 30 or more nursing pads 100.

FIG. 7 is a flow diagram of a method 700 of using the nursing pad 100. The method 700 includes providing 702 the nursing pad 100 including the absorbent pad 102 and the adhesive 104. The adhesive 104 includes the adhesive substance and the backing 112. The method 700 also includes removing 704 the backing 112 to expose the adhesive substance. The method 700 further includes applying 706 the nursing pad 100 to the user's breast by adhering the adhesive substance to the user's breast. The method 700 also includes removing 708 the nursing pad 100 from the user's breast by pulling the nursing pad 100 such that the adhesive substance no longer adheres to the user's breast. The method 700 further includes reapplying 710 the nursing pad 100 to the user's breast by adhering the adhesive substance to the user's breast.

Yet another aspect of the present disclosure relates to a method of making a nursing pad. The method includes applying an adhesive to a first quilted layer of an absorbent pad. The adhesive may be applied in a variety of locations on the first quilted layer. For example, in some implementations, the adhesive is applied in a ring or circular shape. The method also includes applying a backing onto the adhesive to protect the area of the adhesive before use. The backing is applied to the area of adhesive that does not contact the first quilted layer. A user can later remove the backing and affix the adhesive to the user's breast during use. In some implementations, the backing may include a pull tab for later removal of the backing from the adhesive. The method also includes layering at least one additional layer of an absorbent pad on the first quilted layer of an absorbent pad. In some implementations, the at least one additional layer may be numerous (e.g., 4-5) layers (e.g., of organic cotton). The method also includes applying a barrier layer on the at least one additional layer of the absorbent pad. In some implementations, the barrier layer may be a vapor barrier or other material that resists diffusion of moisture and keeps the nursing pad from leaking milk. The method also includes compressing the first quilted layer of an absorbent pad, the at least one additional layer of an absorbent pad, and the barrier layer together. The compressing of the first quilted layer of an absorbent pad, the at least one additional layer of an absorbent pad, and the barrier layer may be performed manually or by machine. In some implementations, the first quilted layer of an absorbent pad, the at least one additional layer of an absorbent pad, and the barrier layer together are stitched together (e.g., with thread or nylon). The stitching may occur around the perimeter of the absorbent pad or in other locations.

This description provides examples, and is not intended to limit the scope, applicability or configuration of the principles described herein. Rather, the ensuing description will provide those skilled in the art with an enabling description for implementing various aspects of the principles described herein. As can be understood by one skilled in the art, various changes may be made in the function and arrangement of elements without departing from the application.

It should be appreciated by a person skilled in the art that one or more aspects of the disclosure may be implemented in a system to additionally or alternatively solve other problems than those described above. Furthermore, aspects of the disclosure may provide technical improvements to “conventional” systems or processes as described herein. However, the description only includes example technical improvements resulting from implementing aspects of the disclosure, and accordingly do not represent all technical improvements provided within the scope of the claims.

It should be noted that these methods describe examples of implementations, and that the operations and the steps may be rearranged or otherwise modified such that other implementations are possible. In some examples, aspects from two or more of the methods may be combined. For example, aspects of each of the methods may include steps or aspects of the other methods, or other steps or techniques described herein. Thus, aspects of the disclosure may provide for consumer preference.

The description set forth herein describes example configurations and does not represent all the examples that may be implemented or that are within the scope of the claims. The term “example” used herein means “serving as an exemplary, instance, or illustration,” and not “preferred” or “advantageous over other examples.” The detailed description includes specific details for the purpose of providing an understanding of the described techniques. These techniques, however, may be practiced without these specific details.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein but is to be accorded the broadest scope consistent with the principles and novel features disclosed herein.

What is claimed is:

1. A nursing pad comprising:
an absorbent pad defining a first side and a second side, wherein the absorbent pad has a contoured shaped configured to correspond to a shape of a user's breast; and
an adhesive attached to the first side of the absorbent pad, wherein the adhesive is configured to adhere the absorbent pad to the user's breast.

2. The nursing pad of claim 1, wherein the absorbent pad comprises a barrier layer attached to the second side of the absorbent pad.

3. The nursing pad of claim 1, wherein the adhesive comprises an adhesive substance and a backing.

4. The nursing pad of claim 3, wherein the adhesive substance comprises a medical grade adhesive.

5. The nursing pad of claim 3, wherein the adhesive substance in an area on the adhesive substance that is not in contact with the first side of the absorbent pad.

6. The nursing pad of claim 3, wherein the adhesive substance and the backing form an adhesive ring on the first side of the absorbent material.

7. The nursing pad of claim 3, wherein the backing comprises a pull tab that enables the user to remove the backing from the adhesive substance.

8. The nursing pad of claim 1, wherein the absorbent pad comprises a barrier layer, a plurality of intermediate layers of absorbent material, and an inner layer configured to be in contact with the user's breast.

9. The nursing pad of claim 8, wherein the inner layer is formed of a quilted organic cotton.

10. A nursing pad comprising:
an absorbent pad defining a first side and a second side, wherein the absorbent pad is formed of a plurality of layers of organic cotton; and
an adhesive attached to the first side of the absorbent pad, wherein the adhesive is configured to adhere the absorbent pad to the user's breast.

11. The nursing pad of claim 10, wherein the absorbent pad comprises a barrier layer attached to the second side of the absorbent pad.

12. The nursing pad of claim 10, wherein the adhesive comprises an adhesive substance and a backing.

13. The nursing pad of claim 12, wherein the adhesive substance comprises a medical grade adhesive.

14. The nursing pad of claim 12, wherein a first area of the adhesive substance is attached to the first side of the absorbent pad and the backing covers a remaining area of the adhesive substance that is not in contact with the first side of the absorbent pad.

15. The nursing pad of claim 12, wherein the adhesive substance and the backing form and adhesive ring on the first side of the absorbent material.

16. The nursing pad of claim 12, wherein the backing comprises a pull tab that enables the user to remove the backing from the adhesive substance.

17. The nursing pad of claim 10, wherein the absorbent pad comprises a barrier layer, a plurality of intermediate layers of absorbent material, and an inner layer configured to be in contact with the user's breast.

18. The nursing pad of claim 17, wherein the inner layer is formed of a quilted organic cotton.

19. A method of using a nursing pad, the method comprising:
providing the nursing pad including an absorbent pad and an adhesive, the adhesive includes an adhesive substance and a backing;
removing the backing to expose the adhesive substance; and
applying the nursing pad to a user's breast by adhering the adhesive substance to the user's breast.

20. The method of claim 19, further comprising removing the nursing pad from the user's breast by pulling the nursing pad such that the adhesive substance no longer adheres to the user's breast.
